(19) United States
(12) Patent Application Publication
(10) Pub. No.: US 2015/0216924 A1
(43) Pub. Date: Aug. 6, 2015

(54) Method of Obtaining Phytochemicals Including Resveratrol and e-Viniferin From Vineyard Waste

(71) Applicant: Ahmad Alkayali, Murrieta, CA (US)

(72) Inventor: Ahmad Alkayali, Murrieta, CA (US)

(21) Appl. No.: 14/170,793

(22) Filed: Feb. 3, 2014

(51) Int. Cl. A61K 36/87; A61K 31/015; A61K 31/05; A61K 31/343

(52) U.S. Cl. CPC A61K 36/87; A61K 31/343; A61K 31/015; A61K 31/05

(57) ABSTRACT

Phytochemicals of the grape, including trans-resveratrol, trans-viniferin, and oligostilibines are obtained from vineyard waste. Also, a dry powder or granular form of food supplement is provided including the phytochemicals obtained from vineyard waste, along with red wine and raw grape juice demonstrates high values of anti-oxidants. A method of making this food supplement includes solvent extraction from vineyard waste, combination with products of the grape fruit, and by vacuum drying without detrimental overheating.

### CROSS REFERENCE TO RELATED APPLICATION

This application is a Continuation-in-Part of U.S. application Ser. No. 11/787,512, filed 17 Apr. 2007, and is also a Continuation-in-Part of U.S. application Ser. No. 12/075,847, filed 14 Mar. 2008, the disclosures of which are incorporated by reference herein to the full extent necessary for a complete and enabling disclosure of the present invention.

### BACKGROUND OF THE INVENTION

### Field of the Invention

This invention relates generally to a method of obtaining valuable phytochemicals, including resveratrol and e-viniferin, which have a beneficial use as a food supplement. The phytochemicals are obtained from waste materials heretofore discarded, burned, or composted as by-products of vineyard fruit production (i.e., from viticulture waste).

Preferably, the food supplement containing the phytochemicals so obtained is presented to consumers as a dry powder, granular, or flake form of food supplement. This food supplement in dry form then can be added conveniently to the consumer's ordinary diet by sprinkling it on other food, or by mixing it into beverages.

More particularly, this invention relates to a food supplement which is a powerful anti-oxidant red grapes, from red wine, and from grape plant parts which are ordinarily discarded by a vineyard operator (i.e., viticulture waste). Importantly, the food supplement is obtained by solvent extraction without the use of elevated temperatures so that the chemical constituents of the food supplement are preserved and are not degraded, oxidized, deteriorated, or de-natured by elevated temperatures.

For example, the plant source materials may include red wine, red grapes, and portions of the red grape plant, including arm portions, cordons, spurs, buds, nodes, cane, tendrils, and even leaves, all of which are ordinarily discarded (i.e., viticulture waste) in the operation of a vineyard. The perennial and high-value parts of the grape plant, such as the fruit (i.e., grapes) root stock, trunk, head, and the arm portions immediately adjacent to the head (which are left on the grape plant at part of the training of the vine and as part of annual vineyard maintenance) are not utilized by this invention. Thus, vineyard operation and fruit production are not at all detrimentally affected by this invention, and only vineyard waste materials usually discarded are utilized in order to obtain a valuable food supplement source according to this invention. Thus, a vineyard's yield of beneficial product is increased, and the cost of this food supplement product is reduced.

### RELATED TECHNOLOGY

Resveratrol is a phytoalexin produced by several plants and sold as a nutritional supplement. It has also been produced by chemical synthesis. A number of beneficial effects, such as anti-cancer, antiviral, neuroprotective, anti-aging, anti-inflammatory and life-prolonging effects have been reported in non-human species. Resveratrol is found in the skin of red grapes and in a constituent of red wine but apparently not in sufficient amounts to explain the so-called French paradox. That paradox relates to the low incidence of coronary heart disease in southern France despite a diet that is high in saturated fats. It has been found that Resveratrol increases the activity of a protein SIRT1, and that this protein significantly increases the lifespan of yeast and mice. Four stilbenes cis and trans resveratrol, and cis and trans piceid are similar and related, and are sometimes analyzed together as a group. Resveratrol produced by plants apparently has anti-fungal properties, and is found in widely varying amounts in grapes (primarily in the skins) and in the roots and stalks of giant knotweed and Japanese knotweed. Muscadine grapes and muscadine wines are also known to contain a good source of resveratrol.

### SUMMARY OF THE INVENTION

In view of the deficiencies of the conventional technology, an object for this invention is to overcome or ameliorate one or more of those deficiencies.

An object of this invention is to obtain Phytochemicals from vineyard waste.

One particularly preferred embodiment of the present invention provides a method of utilizing vineyard waste, including the steps of obtaining a first vineyard waste source material consisting of trimmed off parts of grape vine plant arms, trimmed off cordons, trimmed off nodes, trimmed off shoots/cane, trimmed off tendrils, and grape vine plant leaves. The first vineyard waste source materials are subjected to solvent extraction to provide a liquid including desired chemical constituents from the vineyard waste source material. A second source material includes grape juice. And a third source material includes red wine. The three source materials are combined and blended, and the combined and blended first, second, and third source materials are converted into a dry powder food supplement product which contains beneficial chemical constituents of the grape vine plant.

An additional object for this invention is to provide a dry product food supplement from vineyard waste, grapes, and red wine, containing resveratrol and other beneficial constituents of the grape vine plant and fruit.

The method and advantages of the present invention will be better understood by those of ordinary skill in the art and others upon reading the ensuing specification when taken in conjunction with the appended drawings wherein:

### BRIEF DESCRIPTION OF THE DRAWING FIGURES

FIGS. 1, and 2 are diagrammatic illustrations of grape vines (i.e., grape plants) in various aspects of training the grape vines for vineyard production of fruit (i.e., grapes), and for purposes of clarifying common vineyard terminology;

FIG. 3 is a process flow chart for a method of producing a dry product food supplement from source materials including grape plant source materials, fresh grape juice, and red wine according to this invention.

### DETAILED DESCRIPTION OF A PREFERRED EMBODIMENT

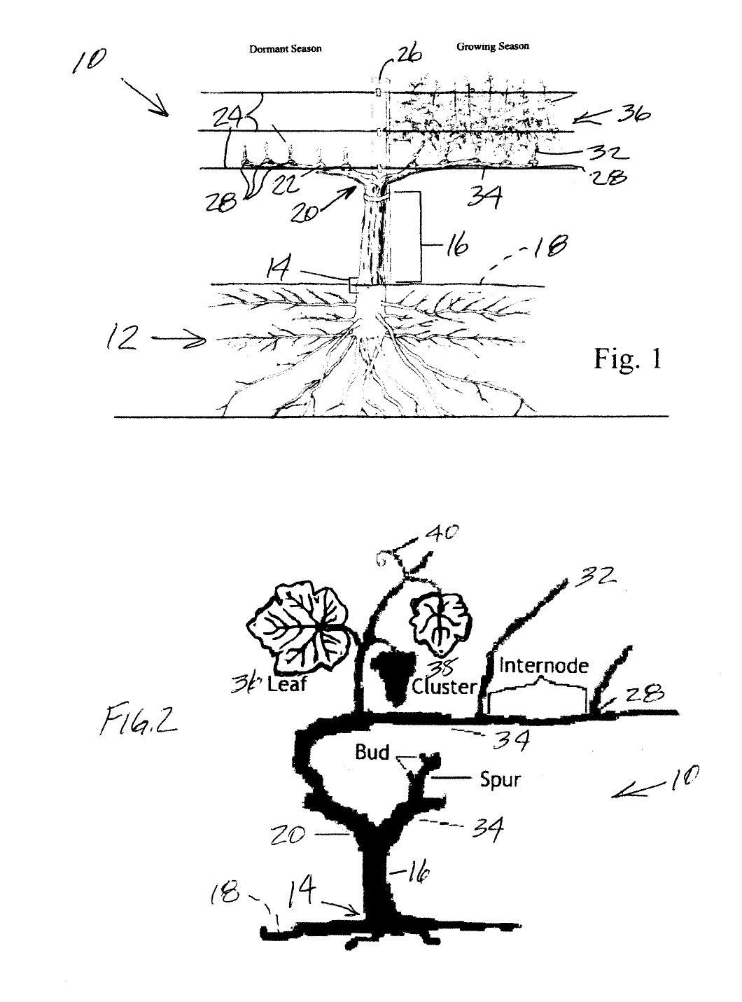

Turning first to FIGS. 1 and 2 in conjunction with one another, and viewing first FIG. 1, a grape vine (i.e., grape plant) 10 is fragmentarily illustrated, with the below-ground root structure 12 (further identified below) of the plant being partially omitted for economy of illustration. As is seen in FIG. 1, the grape vine 10 includes a root structure, or root stock, generally indicated with the arrowed numeral 12. This root stock has a crown 14 at ground level, and leading to a trunk 16 extending upwardly from the level of the ground 18. The trunk leads upwardly to a head 20, and from the head 20 arms 22 of the grape vine extend 10 generally horizontally along wires 24 supported by a post structure 26. The root stock 12, crown 14, trunk 16, head 20, and arms 22 adjacent to the head 20 are perennial. That is, these parts of the grape vine plant endure year after year. But, as is best seen on the left side of FIG. 1, during the dormant season, the grape vine plant is trimmed back by the vineyard operator so that the arms 22 of the vine 10 are truncated. The truncated arms 22 may have several nodes 28, and fruiting spurs 30 may extend from these nodes 28.

On the other hand, during the growing season (seen on the right-hand side of FIG. 1) the grape vine plant 10 produces shoots (i.e., cane) 32 extending from the fruiting spurs 30, and from the arms 22 also grows cordons (i.e., newly growing arm portions) 34 producing additional nodes 28, and additional shoots/cane 32 growing from these additional nodes 28.

Viewing now FIGS. 1 and 2 in conjunction with one another, it is seen that the shoots/cane 32 may give growth to leaves 36, to clusters of grapes 38, and to tendrils 40, the latter being utilized by the grape vine plant 10 to support itself from the wires 24 and possibly from post 26 as well.

Comparing the right-hand side and the left-hand side of FIG. 1, it is seen that at the end of the growing season and after the harvest of grapes 38, the grape vine plant 10 still includes a considerable amount of excess vegetation that must be trimmed back by the vineyard operator in preparation for the dormant season. That is, part of the arms 22, the cordons 34, the nodes 28 of these cordons 34, the cane 32, the leaves 36, and the tendrils 40, are all trimmed away. This vineyard trimming or husbandry prepares the grape vine plants for the next dormant season, and insures that the plants are ready for fruit production during the next growing season. Traditionally, this excess vegetation trimmed from the grape vine 10 at the end of the growing season was considered vineyard waste, and was burned, composted, or was simply sent to land fills. As stated, some of this vineyard waste may have been composted for its use as fertilizer and as a soil conditioner, but that was the extent of value traditionally obtained from this vineyard waste material. Dealing with this vineyard waste has traditionally been a task and burden of vineyard husbandry.

In contrast to traditional vineyard practices, the applicant has discovered that the vineyard waste material identified above (hereinafter referred to with reference numeral 42) may be utilized as an important source material of a valuable food supplement. That is, important human nutritional materials may be obtained from the vineyard waste 42 which heretofore was mostly disposed of or was simply burned.

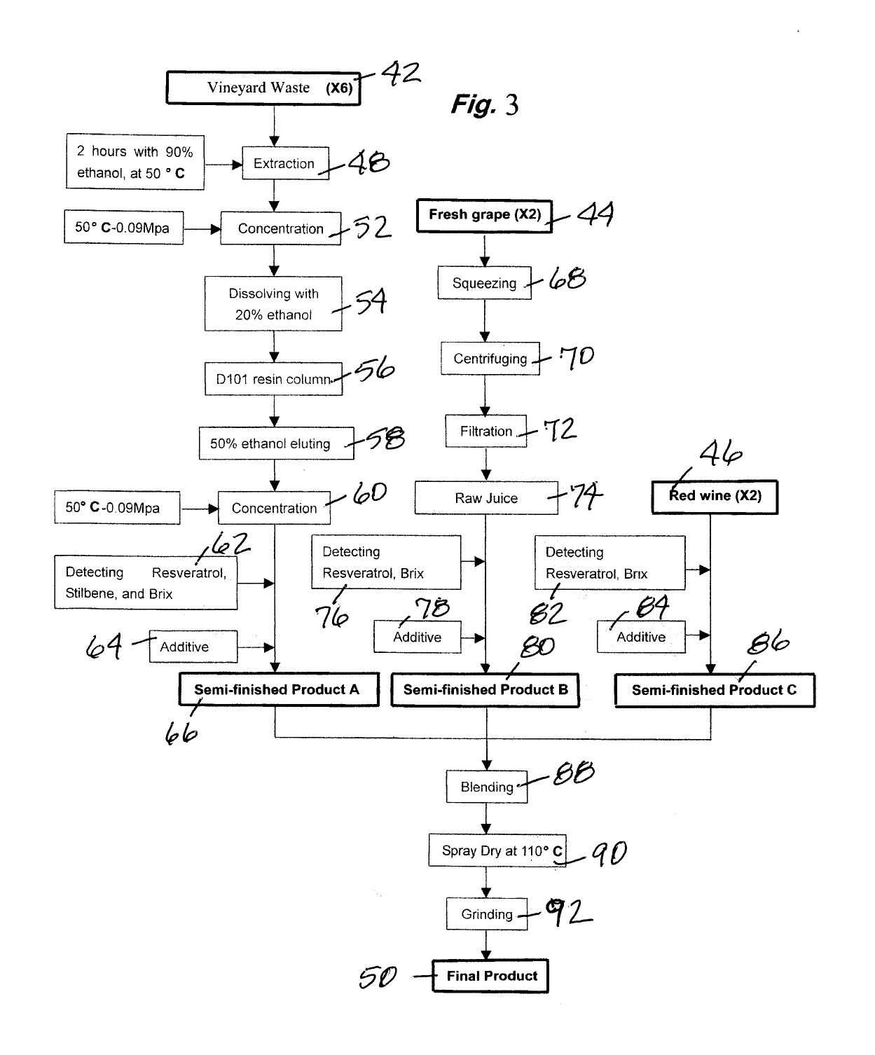

Turning now to FIG. 3, this Figure schematically illustrates a process or method for producing a free-flowing dry crystalline or powder food supplement product 50 using source materials (indicated as 42, 44, and 46). As is indicated on FIG. 3, the source materials 42, 44, and 46 are preferably used in (6:2:2) proportions generally indicated on this Figure, although the invention is not so limited. That is, 6 parts of vineyard waste source material 42, to two parts of source material 44, along with 2 parts of source material 46 are preferably utilized in the process illustrated by FIG. 3. As stated, the source material 42 consists of the vineyard waste materials defined above (i.e., the trimmed off outer portions or parts of the arms 22, the cordons 34, the nodes 28 of these cordons 34, the shoots/cane 32, the leaves 36, and the tendrils 40) (hereinafter refereed to collectively as “vineyard waste”). While the preferred source of this vineyard waste is the red grape, white muscadine grapes plants and parts of these grape plants may be included. It is important to note that this vineyard waste source material 42 includes many materials that are simply trimmed off the grape vines during maintenance of a vineyard, and which historically were thrown into a land fill or burned.

Source material 44 includes fresh grapes, and again red grapes are preferred although other grapes such as the white muscadine grape may be included. Source material 46 includes red wine. It will be noted on FIG. 3, that the process parameters are indicated as included 6 measures of source material 42, and 2 measures of each of the source materials 44 and 46. These measures are indicated to be by weight, although the proportions of the source materials are not critical to obtaining a beneficial product according to this invention. In other words, the invention is not limited to the proportions of source materials indicated on FIG. 1. The vineyard waste source material 42 is most preferably washed to remove debris, and then is ground to a sufficiently small size allowing for efficient solvent extraction before the process of FIG. 3 is begun.

Considering first the processing of the grape cane source material 42, it is seen on FIG. 3 that the vineyard waste source material 42 is treated with a 90 percent ethanol solution at approximately 50° C. for a time of about two hours (step 48). The solution is then drained from the vineyard waste material, and this solution of extraction (also known as liquid #1—i.e., from step 48) is concentrated by vacuum concentration or drying at 0.09 Mpa, and 50° C. (step 52). The resulting semi-dry or dry material of extraction (i.e., from step 50 is dissolved with 20 percent ethanol (step 54), and is then passed along a D101 resin column (step 56). The captured extraction material in the resin column of step 56 is eluted using 50 percent ethanol (step 58). Again, the resulting solution now carrying the desired constituents extracted from the vineyard waste material 42 (i.e., placed back into solution at step 58) is vacuum concentrated or partially dried at 0.09 Mpa, and 50° C. (step 60).

At steps 62 and 64 are illustrated two optional steps, the first (step 30) being an assaying step during which the concentrated or dried material from step 60 is tested for the presence of resveratrol, stilbene, and brix. At step 64 is illustrated the optional addition of additives to the semi-finished product A, which is generally indicated on FIG. 3 with the numeral 66. The semi-finished product A indicated by numeral 66 is a concentrated extract of vineyard waste, containing resveratrol, and stilbene.

Turning now to the processing of the fresh grape source material 44, as is seen on FIG. 3, the fresh grape 44 is squeezed (step 68) to provide fresh grape juice (i.e., from step 68). The fresh grape juice from step 68 is then centrifuged (step 70) and filtered (step 72) to provide raw grape juice (i.e., indicated on FIG. 3 with reference numeral 72). Again, the raw grape juice 72, as was the case with the concentration obtained at step 60 of the first process explained above, may be subjected to two additional optional steps, indicated at 76 and 78. Step 76 is an assaying step during which the raw grape juice from step 74 is tested for the presence of resveratrol, stilbene, and brix. At step 78 is illustrated the optional addition of additives to the semi-finished product B, which is indicated on FIG. 3 with the numeral 80. The semi-finished product B obtained at step 80 is a raw grape juice containing resveratrol, and stilbene.

Now, consider the processing of the red wine source material 46, as is also seen on FIG. 3. This red wine source material 46, as was the case with the concentration indicated at step 60 of the first process explained above, and as with the raw grape juice indicated at step 74 of the second process explained above, may be subjected to two optional steps, indicated at 82 and 84. Step 82 is an assaying step during which the red wine source material 46 is tested for the presence of resveratrol, stilbene, and brix. At step 84 is illustrated the optional addition of additives to the semi-finished product C, which is indicated on FIG. 3 with the numeral 86. The semi-finished product C indicated on FIG. 3 with the numeral 86 is red wine containing resveratrol, and stilbene.

Once the semi-finished Products A (reference numeral 66), B (reference numeral 80), and C (reference numeral 86) are obtained as described above, three additional steps are performed to produce the final product 50. The first of these three additional steps is a blending step indicated at 88. Each of the three semi-finished products A, B, and C are liquid, so this step merely combines and mixes these three constituent materials. Next, the blended semi-finished product from step 88 is spray dried at step 90 at a low pressure (i.e., perhaps as low as 0.09 Mpa), and at an elevated temperature sufficient to result in a dry crystalline or powder product (i.e., perhaps as high a temperature as 110° C. (step 90). It should be noted that although the temperature used at step 90 may appear to be above boiling point of water, in fact the heat of vaporization of the liquid semi-finished product results in the product not fully experiencing this temperature, so that the chemical constituents of the semi-finished product are not deteriorated by the elevated temperature used in step 90. The spray drying step 90 results in a dry flake, chunk, crystalline, or cake-like material. Finally, a grinding step indicated at 92 is employed to insure that there are no lumps or large crystalline formations included in the free-flowing dry powder product 50 produced by this process.

Further to the above, it is to be noted that a typical chemical analysis of the product 50 made as described above will show:

| Trans-resveratrol (HPLC) | 0.32% or 3200 mcg/gm |
|---|---|
| Trans-E-viniferin | 0.64% or 6400 mcg/gm |
| Oligostilibines | 0.80% or 8000 mcg/gm |
| Polyphenols & Oligomeric Proanthocyanidin | 0.88.5% or 8850 mcg/gm |
| (opc)) | |
| Moisture | 4.7% |
| Ashes | 5% |

The balance of the product 50 is essentially polyphenols. With this product 50, a preferred dosing for beneficial human consumption of the product is 1 gram per day. However, the invention is not limited to this level of consumption, and a beneficial result may be obtained with dosing as low as about 200 mcg/day or as high as even 20 grams/day. The product 50 may be taken by mixing it with fruit juice, or in the form of capsules or pressed into tablets. Alternatively, the dry powder product 50 may simply be ingested, or perhaps sprinkled on other foods before they are eaten.

Importantly, the particular levels of beneficial grape plant chemicals identified above are also not limiting on this invention. This is because the finished product identified in FIG. 3, for example, may be further purified to remove a desired portion of the polyphenols, leaving a product which is proportionately higher in concentrations of the trans-resveratrol (HPLC), trans-viniferin, and oligostilibines. For example, the product can be further refined by removal of a desired portion of the polyphenols, it is believed, so that the levels of active ingredients are:

| | |
|---|---|
| Trans-resveratrol (HPLC) | 20% or 200 mg/gm |
| Trans-viniferin | 22% or 220 mg/gm |
| Oligostilibines | 57% or 570 mg/gm |

With this level of purification, the polyphenols are reduced to about 1 percent of the product.

It has been found that a dry food supplement made by drying a moist source material originating from a source selected as outlined above retains a substantial content of the beneficial food values, and that because the mass or quantity of the food supplement (compared to its moist state) is substantially reduced, a consumer of the food supplement need not consume nearly as much of the food supplement as they would have to consume of the source material in order to obtain the desired benefits. And further, the vineyard waste used as a major constituent of this product would not be consumed by humans at all. So, prior to the benefit of this invention being brought to the public, the supplemental food value of this vineyard waste material was simply lost, burned, or discarded.

1. A method of making a food supplement obtained from the grape vine plant, said method comprising steps of:
1) collecting vineyard waste materials consisting of trimmed off parts of grape vine plant arms, trimmed off cordons, trimmed off nodes, trimmed off shoots/cane, trimmed off tendrils, and grape vine plant leaves;
2) collecting fresh grapes, and utilizing said fresh grapes to make grape juice;
3) providing red wine;
4) washing said vineyard waste materials of step #1, and grinding said washed vineyard waste materials to a sufficiently small size allowing for efficient solvent extraction;
5) soaking said vineyard waste materials from step #4 in a solution of ethanol at about 90% strength at approximately 50° C. for a time of about two hours;
6) draining the solution of step #5 away from the vineyard waste materials;
7) concentrating the drained solution of step #6 by vacuum concentration at about 0.09 Mpa, and about 50° C. to provide a semi-dry or dry material of extraction;
8) dissolving the semi-dry or dry material of extraction from step #7 with ethanol at about 20% strength;
9) utilizing a resin column to capture desired chemical constituents from the solution of step #8, and eluting the captured chemical constituents using an ethanol solution at about 50% strength;
10) vacuum concentrating the solution of step #9 at pressure/temperature conditions of about 0.09 Mpa, and 50° C. to provide a semi-finished liquid product including resveratrol and stilbene.
11) combining grape juice (step #2) and red wine (step #3) both containing resveratrol and stilbene with the semi-finished product of step #10; and
12) spray drying the liquid of step #11 at a low pressure and elevated temperature sufficient to result in a dry product.

2. The method of making a food supplement according to claim 1, wherein said method further comprises steps of: utilizing 6 parts of vineyard waste material, to 2 parts grape juice, and to 2 parts red wine (6:2:2 proportions) to make the dry product.

3. The method of making a food supplement according to claim 1, wherein said method further comprises the step of: utilizing vineyard waste material from red grapes.

4. The method of making a food supplement according to claim 1, wherein said method further comprises the step of: utilizing vineyard waste material from white muscadine grapes.

5. The method of making a food supplement according to claim 1, wherein said method further comprises steps of: utilizing red wine made entirely from red grapes.

6. The method of making a food supplement according to claim 1, wherein said method further comprises the step of: utilizing a D101 resin column in step #9.

7. A method of making a grape product food supplement, said method comprising steps of:
providing a first vineyard waste source material consisting of trimmed off parts of grape vine plant arms, trimmed off cordons, trimmed off nodes, trimmed off shoots/cane, trimmed off tendrils, and grape vine plant leaves;
subjecting said first source materials to solvent extraction to provide a liquid including desired chemical constituents from the vineyard waste source material;
providing a second source material including raw grape juice; and
providing a third source material including red wine;
combining and blending said first, said second, and said third source materials; and
converting said combined and blended first, second, and third source materials into a dry powder food supplement product which contains beneficial chemical constituents of the grape vine plant.

8. The method of claim 7, wherein said first source material, said second source material, and said third source material are provided at a ratio of about 6:2:2.

9. The method of claim 7 wherein said first vineyard waste source material is first washed and ground, and is then soaked in an ethanol/water mixture at elevated temperature for a determined time interval to provide a first solution.

10. The method of claim 9, further including the step of vacuum concentrating said first solution obtained according to claim 9.

11. The method of claim 10 further including the step of using in sequence: ethanol dissolution, resin absorption, and ethanol dissolution, followed by another vacuum concentration to provide a liquid semi-finished product from the vineyard waste material.

12. The method of claim 11 further including the steps of mixing the semi-finished product from the vineyard waste material obtained according to claim 11, with grape juice, and with red wine, and
spray vacuum drying the mixture of semi-finished product from the vineyard waste materials, grape juice, and red wine to provide a dry powder food supplement product of the grape vine plant.

13. A method of making a dry food supplement containing phytochemicals from the grape vine plant, said method comprising steps of:
providing a first source material consisting of vineyard waste vegetation;
subjecting said first source materials to solvent extraction to provide a first liquid including desired phytochemical constituents from the vineyard waste source material;
providing a second source material consisting of grape juice; and
providing a third source material consisting of red wine;
combining and blending said first liquid, said second, and said third source materials; and
converting said combined and blended first liquid, said second, and said third source materials into a dry powder food supplement product by spray drying at a low pressure and temperature sufficiently low as to avoid temperature-induced degradation of the phytochemical constituents, so that the resulting dry food supplement contains beneficial phytochemical constituents of the grape vine plant.

14. The method of claim 13, wherein said first source material, said second source material, and said third source material are provided at a ratio of about 6:2:2.

15. The method of claim 13 wherein said first vineyard waste source material is first washed and ground, and is then soaked in an ethanol/water mixture at elevated temperature for a determined time interval to provide a first solution.

16. The method of claim 15, further including the step of vacuum concentrating said first solution obtained according to claim 15.

17. The method of claim 16 further including the step of using in sequence: ethanol dissolution, resin absorption, and ethanol dissolution, followed by another vacuum concentration to provide said first liquid which is then combined with grape juice and red wine.

18. The method of claim 17 further including the step of spray vacuum drying the mixture of said first liquid, grape juice, and red wine at low pressure and a temperature of substantially 110° C. to provide a dry powder food supplement product.
